(19) United States
(12) Patent Application Publication
(10) Pub. No.: US 2010/0305168 A1
(43) Pub. Date: Dec. 2, 2010

(54) PERSONAL-CARE COMPOSITION COMPRISING A CATIONIC ACTIVE

(76) Inventors: Larry Rich Robinson, Loveland, OH (US); Gary Robert Kelm, Cincinnati, OH (US); Denver Michael Faulk, Hyde Park, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/729,593

(22) Filed: Mar. 23, 2010

Related U.S. Application Data
(60) Provisional application No. 61/162,501, filed on Mar. 23, 2009.

(51) Int. Cl. A61K 8/92; A61Q 5/06; A61Q 19/00

(52) U.S. Cl. 514/358; 514/636; 514/561

(57) ABSTRACT

In one embodiment, a personal-care composition in the form of an oil-in-water emulsion comprises an anionic pairing agent, a cationic active, and an anionic thickener. In another embodiment, the anionic pairing agent is pre-formed from the neutralization of an acid with a base. In another embodiment, the anionic pairing agent is formed in situ from the neutralization of an acid with a base. In one embodiment, the molar ratio of the base to the acid is at least about 0.70. In another embodiment, the equivalent ratio of the anionic pairing agent to the cationic active is at least about 0.70. The personal-care composition allows for previously unattainable levels of the cationic active to locate within the oil phase of the emulsion.

### CROSS REFERENCE TO RELATED APPLICATION

This application claims the benefit of Provisional Application Ser. No. 61/162,501, filed Mar. 23, 2009.

### FIELD OF THE INVENTION

The present invention relates to a personal-care composition in the form of an oil-in-water emulsion comprising a cationic active and an anionic thickener. The personal-care composition provides enhanced deliverability of the cationic active to keratinous tissue via the ion-pairing of an anionic pairing agent with the cationic active.

### BACKGROUND OF THE INVENTION

Oil-in-water emulsions offer an ideal platform for personal-care compositions. Anionic thickeners or emulsifiers are widely used in personal-care compositions because of their ability to effectively modify the rheology of the composition, stabilize the emulsion, and provide desired skin-feel benefits. Although cationic thickeners or emulsifiers provide good bioavailability of cationic actives, they may be troublesome if a user applies a cationic-thickener containing product in combination with an anionic-thickener containing product, e.g., moisturizer followed by foundation and/or sunscreen. The cationic thickener and anionic thickener have the potential to bind together and peel off of or produce undesirable prills on the application surface, e.g., on the face. Nonionic thickeners may be used, but few are suitable for personal-care compositions. Accordingly, it is desirable to provide personal-care compositions comprising an anionic thickener.

It is also desirable to provide personal-care compositions comprising a cationic active. Cationic actives may, among other things, reduce hyperpigmented spots on the skin and improve skin barrier function. As will be understood by the skilled artisan, a given component will distribute primarily into either the water or oil phase, depending on the water solubility/dispensability of the component in the composition. For example, if a cationic active resides in the water phase it has been learned that a majority (upwards of 70%) of the cationic active may become bound to and trapped within the swollen anionic thickener in the water phase—thus impeding the delivery and skin bioavailability of the cationic active. For better delivery and bioavailability of the cationic active, it is desirable that the cationic active not be bound and trapped within the anionic thickener. Furthermore, the cationic active may complex with the anionic thickener, forming undesirable solid particles in the product.

There is a need for a personal-care composition able to effectively deliver a cationic active even when an anionic thickener is present.

### SUMMARY OF THE INVENTION

The present invention addresses the needs identified in the Background. In one embodiment, the present invention is directed to a personal-care composition in the form of an oil-in-water emulsion comprising: a) from about 0.0001% to about 20% of an anionic pairing agent; b) from about 0.001% to about 20% of a cationic active; and c) from about 0.001% to about 20% of an anionic thickener. In one embodiment, the anionic pairing agent is pre-formed from the neutralization of an acid with a base, while in another embodiment the anionic pairing agent is formed in situ from the neutralization of an acid with a base. In some embodiments, the molar ratio of the base to the acid is at least about 0.70. In other embodiments, the equivalent ratio of the anionic pairing agent to the cationic active is at least about 0.70. In particular embodiments, the acid is selected from the group consisting of carboxylic acids, alkyl or aryl sulfonic acids, and salts, derivatives, and mixtures thereof.

In another embodiment, the present invention is directed to a method for improving deliverability of a cationic active in the presence of an anionic thickener, comprising the steps of: providing one of the aforementioned personal-care compositions, and applying the composition to keratinous tissue in need of treatment.

In another embodiment, the present invention is directed to a method for improving or regulating keratinous tissue condition, comprising the steps of: providing one of the aforementioned personal-care compositions; and applying the composition to keratinous tissue in need of treatment.

In another embodiment, the present invention is directed to a method for improving deliverability of a cationic active in the presence of an anionic thickener, comprising the steps of: a) preparing an oil phase and a water phase; b) adding from about 0.001% to about 20% of a cationic active to either the oil phase or the water phase; c) adding from about 0.0001% to about 20% of an carboxylic acid to either the oil phase or the water phase; d) adding a base to either the oil phase or the water phase; e) mixing the water phase and the oil phase to form an emulsion; and f) adding to the emulsion from about 0.001% to about 20% of an anionic thickener; wherein the molar ratio of the base to the acid is at least about 0.70, and wherein the equivalent ratio of the anionic pairing agent to the cationic active is at least about 0.70.

### BRIEF DESCRIPTION OF THE DRAWINGS

FIG. 1 is a graph plotting the percent of cationic active in the oil phase for three formulations.

### DETAILED DESCRIPTION OF THE INVENTION

All percentages are by weight of the personal-care composition, unless otherwise specified. All ratios are weight ratios, unless specifically stated otherwise. All such weights as they pertain to listed ingredients are based on the active level and do not include carriers or by-products that may be included in commercially available materials, unless otherwise specified. The number of significant digits conveys neither limitation on the indicated amounts nor on the accuracy of the measurements. All measurements are understood to be made at about 25° C. and at ambient conditions, where “ambient conditions” means conditions under about one atmosphere of pressure and at about 50% relative humidity. All ranges are inclusive and combinable; therefore, every range given throughout this specification will include every narrower range that falls within such broader range as if such narrower ranges were all expressly written herein.

“Personal-care composition,” as used herein, means compositions suitable for topical application on mammalian keratinous tissue. Compositions of the present invention may be used in skin-care, cosmetic, and hair-care products; non-limiting uses of which include antiperspirants, deodorants, lotions (e.g. hand lotion and body lotion), skin-care products (e.g., face and neck lotions, serums, sprays), sunless tanners, cosmetics (e.g., foundation, concealer, blush, lipstick, lip gloss), depilatories, shampoos, conditioning shampoos, hair conditioners, hair dyes, body washes, moisturizing body washes, shower gels, skin cleansers, cleansing milks, hair and body washes, in-shower body moisturizers, pet shampoos, shaving preparations, after-shaves, razor moisturizing/lubricating strips, razor shave-gel bars, bar soaps, cleansing products, feminine-care products, oral-care products, and baby-care products. The methods of using any of the aforementioned compositions are also included within the meaning of personal-care composition.

“Keratinous tissue,” as used herein, refers to keratin-containing layers disposed as the outermost protective covering of mammals which includes, but is not limited to, skin, hair, and nails.

“Regulating keratinous tissue condition,” as used herein, includes prophylactically regulating and/or therapeutically regulating keratinous tissue condition. Regulating keratinous tissue condition may involve one or more of the following benefits: thickening (i.e., building the epidermis and/or dermis layers of the skin and/or the subcutaeous layers such as fat and muscle and where applicable the keratinous layers of the nail and hair shaft) to reduce atrophy (e.g., of the skin); increasing the convolution of the dermal-epidermal border; decreasing non-melanin skin discoloration such as under eye circles, blotching (e.g., uneven red coloration due to, e.g., rosacea) (hereinafter referred to as “red blotchiness”), and sallowness, decreasing discoloration caused by telangiectasia or spider vessels, decreasing discolorations due to melanin (e.g., pigment spots, age spots, uneven pigmentation) and other chromophores in the skin (e.g., lipofuscin, protein crosslinks such as those that occur with glycation, and the like). Regulating skin condition involves improving skin appearance and/or feel. As used herein, prophylactically regulating skin condition includes delaying, minimizing and/or preventing visible and/or tactile discontinuities in skin (e.g., texture irregularities, fine lines, wrinkles, sagging, stretch marks, cellulite, puffy eyes, and the like in the skin which may be detected visually or by feel). As used herein, therapeutically regulating skin condition includes ameliorating (e.g., diminishing, minimizing and/or effacing) discontinuities in skin.

“Derivatives,” as used herein, means ester, ether, amide and/or salt derivatives of the relevant compound.

It has been found that a desired amount of acid, when combined with a desired amount of base, forms an anionic pairing agent capable of forming an ion pair with the cationic active. The anionic pairing agent complexes, or forms an ion pair, with the cationic active, improving the lipophilicity. This enables the cationic active to shift from the water phase to the oil phase of the emulsion, thereby decreasing or preventing its interaction with the anionic thickener, which typically resides in the water phase of the emulsion. The ion pair will more readily dissociate on skin versus an ion pair formed with an anionic polymer and therefore enhance delivery of the active into the skin. Upon application to the skin, the ion pair dissociates, allowing the cationic active to partition into and diffuse across the keratinous tissue.

Exceeding a specified mole ratio of base to acid in the composition yields unexpected results. It has been found that more anionic pairing agent is created, leading to a greater percentage of cationic active in the oil phase, thus increasing the deliverability of the cationic active. Typically, base is used in a composition as a pH adjuster. Excess base can throw off the pH of the composition, which is highly undesirable in personal-care compositions. Applicants, on the other hand, are using the base for the formation of the anionic pairing agent. Not only does the anionic pairing agent inhibit the charge attraction between the cationic active and the anionic thickener, it also aids in the transfer of the cationic active from the water phase to the oil phase by increasing the hydrophobicity of the cationic active.

### I. PERSONAL-CARE COMPOSITION

The personal-care compositions of the present invention may be used to improve the deliverability of a cationic active in the presence of an anionic thickener. The compositions of the present invention may also be used for topical application to regulate keratinous tissue condition. The compositions of the present invention, including the essential and optional components thereof, are described in detail hereinafter.

The personal-care composition of the present invention is an oil-in-water emulsion comprising a continuous water phase and a discontinuous oil phase. Suitable emulsions may have a wide range of viscosities, depending on the desired product form. In certain embodiments, the personal-care composition may have a viscosity of from about 1,000 cps (centipoise) to about 1,000,000 cps, or from about 5,000 cps to about 500,000 cps, or from about 10,000 cps to about 200,000 cps, or from about 15,000 cps to about 75,000 cps. For example, viscosities may be measured on a Brookfield viscometer using a T-C bar spindle with a heliopath setting at 5 rpm at 25° C. In particular embodiments, the personal-care composition has a pH of from about 3 to about 9, or from about 4 to about 7.

The personal-care composition may comprise at least about 2% of an oil phase. The personal-care composition may comprise from about 2% to about 75%, or from about 5% to about 35%, or from about 10% to about 30%, by weight of the composition, of an oil phase. In particular embodiments, the oil phase of the present invention may comprise silicone oils, non-silicone oils such as hydrocarbon oils, esters, ethers, the like, and mixtures thereof. Suitable non-silicone oils include, but are not limited to, isohexadecane, isopropyl isostearate, and mixtures thereof. Suitable silicone oils include, but are not limited to, polyalkysiloxanes, cyclic polyalkylsiloxanes, polyalkylarylsiloxanes and emulsifying and non-emulsifying silicone elastomers.

The personal-care composition may comprise at least about 25% of a water phase. The personal-care composition may comprise from about 25% to about 98%, or from about 65% to about 95%, or from about 70% to about 90%, by weight of the composition, of a water phase. The water phase typically comprises water. In some embodiments, the water phase may be comprised entirely of water. In other embodiments, the water phase may comprise components other than water (i.e., non-water components), including, but not limited to, water-soluble moisturizing agents, conditioning agents, anti-microbials, humectants, and/or other water-soluble skin care actives, to impart an increased benefit to the keratinous tissue. In one embodiment, the water phase of the personal-care composition comprises a humectant such as glycerin and/or other polyols. In a particular embodiment, the personal-care composition is substantially water-free.

In one embodiment, the composition comprises an emulsifier. In certain embodiments, the personal-care composition may comprise from about 0.05% to about 20%, or from about 0.1% to about 10%, by weight of the composition, of total emulsifier. Emulsifiers may be nonionic, anionic or cationic. Non-limiting examples of emulsifiers are disclosed in U.S. Pat. No. 3,755,560, U.S. Pat. No. 4,421,769, and McCutcheon's Detergents and Emulsifiers, North American Edition and International Edition, pages 235-246 (1993).

In one embodiment, the present invention relates to a personal-care composition in the form of an oil-in-water emulsion comprising an anionic pairing agent, a cationic active, and an anionic thickener. Personal-care compositions with this particular combination of ingredients enable the cationic active to locate in the oil phase, where it is more readily delivered to the consumer than if the active is located as a non-ion pair in the water phase, as is the current standard. This unexpected availability allows for better delivery of the cationic active to the consumer. High levels of cationic active in the oil phase were previously unachievable with compositions comprising anionic thickeners. In one embodiment, at least 25%, or at least 50%, or at least 65%, or at least 90%, by weight of the total cationic active, is measurable in the oil phase of the personal-care composition.

A. Cationic Active

The personal-care composition of the present invention comprises a cationic active. “Cationic active,” as used herein, means an ingredient comprising a positive charge which aids in regulating keratinous tissue condition. In various aspects, the cationic active may increase skin tone, improve skin texture, inhibit destruction of collagen, act as a protease inhibitor, condition hair, or otherwise regulate keratinous tissue condition. In one embodiment, the personal-care composition comprises at least about 0.001%, by weight of the composition, of a cationic active. In certain embodiments, the personal-care composition comprises from about 0.001% to about 20%, or from about 0.01% to about 15%, or from about 0.1% to about 10%, by weight of the composition, of a cationic active.

Suitable cationic actives include, but are not limited to, a hexamidine compound, cetylpyridinium chloride, and amino acids. As used herein, a “hexamidine compound” means a salt or cationic derivative of a compound having the formula:

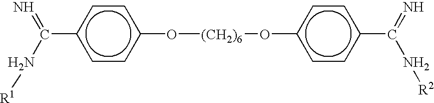

wherein R¹ and R² are hydrogen. In one embodiment, the hexamidine compound may be the salt hexamidine diisethionate.

Cetylpyridinium chloride is a cationic quaternary ammonium. Other suitable cationic quaternary ammoniums that may be cationic actives include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms (typically methyl or ethyl groups). Dodecyl trimethyl ammonium bromide, tetradecylpyridinium chloride, domiphenbromide, N-tetradecyl-4-ethyl pyridinium chloride, dodecyl dimethyl (2-phenoxyethyl)ammonium bromide, benzyl dimethylstearyl ammonium chloride, quaternized 5-amino-1,3-bis(2-ethyl-hexyl)-5-methyl hexahydropyrimidine, benzalkonium chloride, benzethonium chloride and methyl benzethonium chloride are exemplary of typical quaternary ammonium agents. Other compounds are bis-4-(R-amino)-1-pyridinium alkanes as disclosed in U.S. Pat. No. 4,206,215.

As used herein, “amino acid” means a molecule comprising both carboxyl and amine functional groups. Amino acids may be classified as essential and nonessential. Suitable essential amino acids include, but are not limited to, isoleucine, leucine, lysine, methionine, phenylalanine, threonine, tryptophan, valine, and combinations thereof. Suitable nonessential amino acids include, but are not limited to, alanine, asparagine, aspartate, cysteine, glutamate, glutamine, glycine, proline, serine, tyrosine, arginine, histidine, and combinations thereof.

B. Anionic Thickener

The personal-care composition of the present invention comprises an anionic thickener. The personal-care composition comprises from about 0.001% to about 20%, or from about 0.01% to about 15%, or from about 0.1% to about 10%, by weight of the composition, of an anionic thickener.

Suitable anionic thickeners include, but are not limited to, cross-linked acrylic acid-vinyl ester copolymer; sodium polyacrylate; acrylic acid/VP crosspolymer; acrylates/aminoacrylates/C10-30 alkyl PEG-20 itaconate copolymer; acrylates/steareth-20 itaconate copolymer; acrylates/ceteth-20 itaconate copolymer; dehydroxanthan gum; caprylic/capric triglyceride/sodium acrylates copolymer; sodium polyacrylate/hydrogentated polydecence/PPG-5 laureth-5; polyacrylamide/C13-14/laureth-7; polyacrylate 13/polyisobutene/polysorbate 20; acylamide ammonium acrylate copolymer/polyisobutene/polysorbate 20; sodium acrylate/sodium acryloyldimethyl taurate copolymer/polyisobutene/capryl)-capryl glucoside; sodium acrylate/sodium acryloyldimethyl taurate copolymer/isohexadecane/polysorbate 80; hydroxyethyl acrylate/sodium acryloyldimethyl taurate copolymer/squalane/polysorbate 60; hydroxyethyl acrylate/sodium acryloyldimethyl taurate copolymer/isohexadecane/polysorbate 60; caprylic/capric triglyceride/ammonium acryloyldimethyltaurate/beheneth-25 methacrylate cross polymer; ammonium acryloyldimethyl-taurate/VP copolymer; caprylic/capric triglyceride/ammonium acrylolydimethyltaurate/beheneth-25 methacrylate crosspolymer; ammonium acryloyldimethyltaurate/beheneth-25 methacrylate crosspolymer; caprylic/capric triglyceride/ammonium acryloyldimethyltaurate/VP copolymer/trilaureth-4 phosphate/polyglyceryl-2 sesquiisostearate; sodium polyacrylate/C13-14 isoparaffin/trideceth-6; sodium polyacrylate/hydrogenated polydecence/trideceth-6; and hydrophobically-modified alkali soluble polymer emulsion. Preferred thickeners include sodium polyacrylate; sodium polyacrylate/hydrogentated polydecence/PPG-5 laureth-5; sodium acrylate/sodium acryloyldimethyl taurate copolymer/isohexadecane/polysorbate 80 (Simulgel EG from Seppic Corporation, Fairfield, N.J.); hydroxyethyl acrylate/sodium acryloyldimethyl taurate copolymer/squalane/polysorbate 60 (Simulgel NS from Seppic Corporation, Fairfield, N.J.); hydroxyethyl acrylate/sodium acryloyldimethyl taurate copolymer/isohexadecane/polysorbate 60 (Simulgel INS from Seppic Corporation, Fairfield, N.J.); polyacrylamide/C13-14/laureth-7 (e.g., SEPIGEL 305 from Seppic Corporation, Fairfield, N.J.); and sodium polyacrylate/C13-14 isoparaffin/trideceth-6 (e.g., Cosmedia ATH from Cognis).

Further suitable anionic thickeners include, but are not limited to, carboxylic acid polymers and polyacrylamide polymers. Carboxylic acid polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Polymers useful in the present invention are more fully described in U.S. Pat. No. 5,087,445, to Haffey et al, issued Feb. 11, 1992; U.S. Pat. No. 4,509,949, to Huang et al, issued Apr. 5, 1985; U.S. Pat. No. 2,798,053, to Brown, issued Jul. 2, 1957; and in CTFA International Cosmetic Ingredient Dictionary, Fourth Edition, 1991, pp. 12 and 80.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol® 1342, Carbopol® 1382, Pemulen TR-1, and Pemulen TR-2, from B.F. Goodrich. In other words, examples of carboxylic acid polymer thickeners useful herein are those selected from the group consisting of carbomers, acrylates/C₁₀-C₃₀ alkyl acrylate crosspolymers, and mixtures thereof.

Suitable polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc., (Patterson, N.J.).

C. Anionic Pairing Agent

The personal-care composition of the present invention comprises an anionic pairing agent. “Anionic pairing agent,” as used herein, means a non-polymeric ingredient or combination of ingredients which comprises a negative charge. Adding an anionic pairing agent to the personal-care composition promotes the formation of ion pairs between the cationic active and the anionic pairing agent and prevents the formation of ion pairs between the cationic active and the anionic thickener. The anionic pairing agent forms an ion pair with the cationic active and pulls the active from the water phase of the emulsion into the oil phase of the emulsion, if the cationic active starts in the water phase. The anionic pairing agent complexes, or forms an ion-pair complex, with the cationic active, thus mitigating its interaction with the anionic thickener.

The amount of anionic pairing agent necessary to form an ion-pair complex with the cationic active may depend on the specific active chosen. For example, if the anionic pairing agent, e.g., stearate, has one negative charge, and the cationic active, e.g., hexamidine diisethionate, has two positive charges, the molar ratio of anionic pairing agent to cationic active should be about 2:1 in order to completely complex with the cationic active. Or, if the anionic pairing agent has one negative charge, and the cationic active has one positive charge, the molar ratio of anionic pairing agent to cationic active is preferably 1:1. The personal-care composition may comprise from about to about 0.0001% to about 20%, or from about 0.001% to about 15%, or from about 0.01% to about 10%, by weight of the composition, of an anionic pairing agent.

One of ordinary skill in the art would choose the correct molar ratio, or “equivalent ratio,” of the anionic pairing agent to the cationic active in order to convert at least 70% and preferably 100% of the particular cationic active to an ion pair. In one embodiment, the amount of anionic pairing agent may be such that the cationic active is completely complexed, for instance, wherein the equivalent ratio of the anionic pairing agent to the cationic active is about 1:1. In another embodiment, the equivalent ratio may be such that the cationic active is not completely complexed, but still having a higher ratio of ion pair than previously in the art, for example, wherein the equivalent ratio of the anionic pairing agent to the cationic active is at least about 0.70, at least about 0.80, or at least about 0.90.

In some embodiments, the anionic pairing agent may be pre-formed from the neutralization of an acid with a base. For example, the anionic pairing agent may comprise a carboxylate, such as sodium stearate, sodium salicylate, potassium benzoate, or sodium lauryl sulfate.

In other embodiments, the anionic pairing agent may be formed in situ from the neutralization of an acid with a base. The anionic pairing agent may be formed by any combination of acids and bases discussed below. For example, the anionic pairing agent may comprise sodium hydroxide and stearic acid; the resultant sodium stearate is the anionic pairing agent.

To prepare an effective anionic pairing agent, the molar ratio of base to acid is at least about 0.65, or 0.70, or 0.80, or 0.90. In another embodiment, the molar ratio of base to acid is about 1:1 or greater. For example, with a dicarboxcylic acid, the molar ratio of base to acid may be about 2:1 or greater.

The composition may comprise from about 0.0001% to about 10%, or from about 0.001% to about 5%, or from about 0.01% to about 1%, by weight of the composition, of a base. “Base,” as used herein, means a substance that may accept protons or otherwise neutralize an acid.

Suitable bases include, but are not limited to, triethylamine, triethanolamine, sodium hydroxide, potassium hydroxide, ammonium hydroxide, calcium hydroxide, magnesium hydroxide, and combinations thereof.

The composition may comprise from about 0.0001% to about 10%, or from about 0.001% to about 5%, or from about 0.01% to about 1%, by weight of the composition, of an acid. “Acid,” as used herein, means a substance that may donate protons or otherwise neutralize a base. Suitable acids include carboxylic acids, alkyl or aryl sulfonic acids, and salts, derivatives, and mixtures thereof.

Suitable carboxylic acids include, but are not limited to, oleic acid, stearic acid, propionic acid, hexanoic acid, benzoic acid, octadecenedioic acid (Arlatone Dioic DCA), salicylic acid, and retinoic acid. Carboxylic acid salts and derivatives thereof of the present invention correspond to the formula:

RCO₂X

wherein X is Na, K, Mg, Mn, Zn, Cu, triethanolamine, diethanolamine, ammonium, quaternary alkyl ammonium); R is C1-C20 straight or branched alkyl or aryl groups. As used herein, alkyl means carbon containing chains that may be straight or branched or cyclic, substituted or unsubstituted, saturated or monounsaturated or polyunsaturated.

Example carboxylic acid salts include, but are not limited to, salts of hydroxy acids (e.g., salicylic acid, glycolic acid, lactic acid, 3-hydroxy benzoic acid, 4-hydroxy benzoic acid, 2-hydroxybutanoic acid, 2-hydroxypentanoic acid), 2-hydroxyhexanoic acid keto acids (e.g., pyruvic acid), phytic acid, glycyrrhetic acid, glycyrrhetinic acid, cis-retinoic acid, trans-retinoic acid, lipoic acid, azelaic acid, arachidonic acid, lysophosphatidic acid, and salts of amino acids (e.g. undecylenoyl phenylalanine (e.g. Sepiwhite MSH), and mixtures thereof.

One suitable alkyl or aryl sulfonic acid is phenylbenzimidazole sulfonic acid (PBSA).

D. Additional Actives

The composition of the present invention may comprise one or more actives in addition to the cationic active. Particularly suitable actives include sugar amines, vitamin B₃ compounds, vitamins, peptides, and sunscreens.

1. Sugar Amine

The compositions of the present invention may comprise a sugar amine, also known as amino sugars, and their salts, isomers, tautomers and derivatives. As used herein, “sugar amine” refers to a pure sugar amine compound or a mixture of sugar amine compounds (e.g., extracts from natural sources or mixtures of synthetic materials) of synthetic or natural origin, including its isomers, tautomers, salts, and derivatives. Examples of sugar amines useful herein include glucosamine, N-acetyl glucosamine, mannosamine, N-acetyl mannosamine, galactosamine, N-acetyl galactosamine, their isomers (e.g., stereoisomers), and their salts (e.g., HCl salt). In one embodiment, the sugar amine is glucosamine, or D-glucosamine and N-acetyl glucosamine, or N-acetyl-D-glucosamine. Additionally, combinations of two or more sugar amines may be used. The composition may comprise from about 0.01% to about 15%, alternatively from about 0.1% to about 10%, and alternatively from about 0.5% to about 5%, of a sugar amine.

2. Vitamin B₃ Compound

The compositions of the present invention may include a vitamin B₃ compound. As used herein, “vitamin B₃ compound” means a compound having the formula:

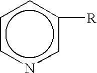

wherein R is —CONH₂ (i.e., niacinamide), —COOH (i.e., nicotinic acid) or —CH₂OH (i.e., nicotinyl alcohol); derivatives thereof; and salts of any of the foregoing. Vitamin B₃ compounds are particularly useful for regulating skin condition as described in U.S. Pat. No. 5,939,082. In one embodiment, the vitamin B₃ compound is niacinamide. The composition may comprise from about 0.001% to about 20%, or from about 0.1% to about 10%, or from about 0.5% to about 7%, by weight of the composition, of a vitamin B₃ compound.

3. Vitamin

The composition of the present invention may comprise one or more vitamins, for example, to provide antioxidant and/or other nutritional benefits to the skin. Herein, “vitamin” means vitamins, pro-vitamins, and their salts, isomers and derivatives. The vitamins may include water soluble vitamins, for example, nicotinic acid, C1-C18 nicotinic acid esters, and nicotinyl alcohol; B₆ compounds, such as pyroxidine; and B₅ compounds, such as panthenol, or “pro-B₅”); and Vitamin C compounds, including ascorbyl esters of fatty acids, and ascorbic acid derivatives, for example, ascorbyl glucoside, magnesium ascorbyl phosphate, sodium ascorbyl phosphate, and ascorbyl sorbate; and mixtures thereof. The vitamins also may include those exhibiting limited solubility in water, such as Vitamin A compounds, and all natural and/or synthetic analogs of Vitamin A, including retinoids, carotenoids, and other compounds which possess the biological activity of Vitamin A; Vitamin D compounds; Vitamin E compounds, or tocopherol, including tocopherol sorbate, tocopherol acetate, other esters of tocopherol; Vitamin K compounds; and mixtures thereof. The composition may comprise from about 0.0001% to about 20%, or from about 0.01% to about 15%, or from about 0.1% to about 10%, by weight of the composition, of a vitamin.

4. Peptide

The compositions of the present invention may include a peptide. As used herein, “peptide” refers to peptides containing ten or fewer amino acids and their derivatives, isomers, and complexes with other species such as metal ions (e.g., copper, zinc, manganese, magnesium, and the like). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, and Cu-histidine-glycine-glycine (Cu-HGG, also known as IAMIN®). A further example includes carnosine (beta-alanine-histidine). Preferred peptides include PROMATRIXYL, comprising palmitoyl pentapeptide-3 and PALESTRINA, comprising palmitoyl dipeptide-7, both available from Croda Inc. The composition may comprise from about 1×10⁻⁶% to about 20%, or from about 1×10⁻⁵% to about 10%, or from about 1×10⁻⁴% to about 5%, by weight of the composition, of a peptide.

5. Sunscreen

The compositions of the subject invention may comprise one or more sunscreen actives. As used herein, “sunscreen active” refers to oil-soluble sunscreens, insoluble sunscreens, and water-soluble sunscreens. Non-limiting examples of suitable oil-soluble sunscreens are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93 and include benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, and derivatives and mixtures thereof. Non-limiting examples of suitable insoluble sunscreens include methylene bis-benzotriazolyl tetramethylbutyl-phenol, titanium dioxide, zinc cerium oxide, zinc oxide, and derivatives and mixtures thereof. Non-limiting examples of suitable water-soluble sunscreens include phenylbenzimidazole sulfonic acid (PBSA), terephthalylidene dicamphor sulfonic acid, (Mexoryl™ SX), benzophenone-4, benzophenone-5, benzylidene camphor sulfonic acid, cinnamidopropyl-trimonium chloride, methoxycinnamido-propyl ethyldimonium chloride ether, disodium bisethylphenyl triaminotriazine stilbenedisulfonate, disodium distyrylbiphenyl disulfonate, disodium phenyl dibenzimidazole tetrasulfonate, methoxycinnamido-propyl hydroxysultaine, methoxycinnamido-propyl laurdimonium tosylate, PEG-25 PABA (p-aminobenzoic acid), polyquaternium-59, TEA-salicylate, and salts, derivatives and mixtures thereof. The composition may comprise from about 0.0001% to about 30%, or from about 0.01% to about 20%, or from about 0.1% to about 10%, by weight of the composition, of a vitamin.

6. Retinoid

The compositions of this invention may comprise a safe and effective amount of a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably selected from retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), or mixtures thereof. In one embodiment, the retinoid is retinyl propionate. The composition may comprise from about 0.001% to about 10%, or from 0.01% to about 1%, or from about 0.01% to about 0.5%, by weight of the composition, of a retinoid.

7. Other Actives

Other suitable actives include, but are not limited to, oil control agents, N-acyl amino acid compounds, tanning actives, anti-acne actives, desquamation actives, anti-cellulite actives, chelating agents, skin lightening agents, flavonoids, protease inhibitors, tyrosinase inhibitors, non-vitamin antioxidants and radical scavengers, preservatives, hair growth regulators, anti-wrinkle actives, anti-atrophy actives, minerals, phytosterols and/or plant hormones, anti-inflammatory agents, antimicrobials, and antifungals. Further suitable actives include caffeine; tea extracts, e.g. white tea extract and green tea extract; ginseng; cucumber extract; rosehip oil; date palm kernel extract; witch hazel extract; dill extract; tetrahydrocurcmin; turmerone; and other natural or botanical compounds. Many of these actives are provided in further detail in U.S. Application Publication Nos. US2006/0275237A1, US2004/0175347A1, and US2006/0263309A1.

E. Optional Ingredients

1. Particulate Material

The compositions of the present invention may comprise from about 0.001% to about 40%, by weight of the composition, of one or more particulate materials. Non-limiting examples of suitable powders include inorganic powders (for example, iron oxides, titanium dioxides, zinc oxides, silica), organic powders, composite powders, optical brightener particles, and mixtures of any of the foregoing. These particulates can, for instance, be platelet shaped, spherical, elongated or needle-shaped, or irregularly shaped; surface coated or uncoated; porous or non-porous; charged or uncharged; and can be added to the current compositions as a powder or as a pre-dispersion. In one embodiment, the particulate material is hydrophobically coated.

Suitable organic powder particulate materials include, but are not limited, to polymeric particles chosen from the methylsilsesquioxane resin microspheres, e.g., Tospearl™ 145A, (Toshiba Silicone); microspheres of polymethylmethacrylates, e.g., Micropearl™ M 100 (Seppic); the spherical particles of crosslinked polydimethylsiloxanes, e.g., Trefil™ E 506C or Trefil™ E 505C (Dow Corning Toray Silicone); spherical particles of polyamide, e.g., nylon-12, and Orgasol™ 2002D Nat C05 (Atochem); polystyrene microspheres, e.g., Dyno Particles, sold under the name Dynospheres™, and ethylene acrylate copolymer, sold under the name FloBead™ EA209 (Kobo); aluminum starch octenylsuccinate, e.g., Dry Flo™ (National Starch); polyethylene particulates, e.g., Microthene™ FN510-00 (Equistar) and Micropoly° 220L (Micro Powders, Inc.); microspheres of polypropylene, e.g., Mattewax™ 511 (Micro Powders, Inc.); silicone resin; polymethylsilsesquioxane silicone polymer; platelet shaped powder made from L-lauroyl lysine; and mixtures thereof.

The composition of the present invention further may comprise interference pigments, including hydrophobically-modified interference pigments. Herein, “interference pigments” means thin, plate-like layered particles having two or more layers of controlled thickness. The layers have different refractive indices that yield a characteristic reflected color from the interference of typically two, but occasionally more, light reflections, from different layers of the plate-like particle. One example of interference pigments are micas layered with about 50-300 nm films of TiO₂, Fe₂O₃, silica, tin oxide, and/or Cr₂O₃ and include pearlescent pigments. Interference pigments are available commercially from a wide variety of suppliers, for example, Rona (Timiron™ and Dichrona™), Presperse (Flonac™), Englehard (Duochrome™), Kobo (SK-45-R and SK-45-G), BASF (Sicopearls™) and Eckart (Prestige™). In one embodiment, the average diameter of the longest side of the individual particles of interference pigments is less than about 75 microns, and alternatively less than about 50 microns.

Non-limiting examples of suitable colorants include iron oxides, ferric ammonium ferrocyanide, manganese violet, ultramarine blue, and chromium oxide, phthalocyanine blue and green pigment, encapsulated dyes, inorganic white pigments, for example TiO₂, ZnO, or ZrO₂, FD&C dyes, D&C dyes, and mixtures thereof.

2. Inorganic Sunscreens

The composition further may comprise from about 0.001% to about 10%, and alternatively from about 0.1% to about 5%, of an inorganic and/or oil-insoluble sunscreen. Non-limiting examples of suitable insoluble sunscreens include methylene bis-benzotriazolyl tetramethylbutyl-phenol, titanium dioxides, zinc cerium oxides, zinc oxides, and derivatives and mixtures thereof.

3. Non-polar Emollient

The composition of the present invention may comprise from about 2% to about 70%, and alternatively 30% to about 50% of a non-polar emollient. Non-limiting examples of suitable non-polar emollients include silicone oils, hydrocarbon oils, and mixtures thereof. Useful non-polar emollients in the present invention include natural, synthetic, saturated, unsaturated, straight chained, branched chained, linear, cyclic, aromatic, volatile, and non-volatile non-polar emollients, and mixtures thereof.

Non-limiting examples of suitable non-polar hydrocarbons oils include mineral oils and branched chain hydrocarbons (such as commercially available, for example, under the tradenames Permethyl™ (Permethyl Corporation™) and Isopar™ (Exxon™)). Non-limiting examples of suitable non-polar silicone oils include linear and cyclic dimethicones. Commercially available examples of these types of silicones include the Dow Corning 200 series, Dow Corning 344, and Dow Corning 345 (all available from Dow Corning™ Corp.); and SF1202, SF1204, and the Viscasil™ series (all available from the G.E. Silicones™). Additional non-polar silicone oils include alkyl (for example, 2 carbons to 30 carbons) and aryl (for example, phenyl or styrenyl) substituted silicones, including by not limited to phenyl methicone, phenyl dimethicone, phenyl trimethicone, diphenyl dimethicone, phenylethyl dimethicone, hexyl dimethicone, lauryl dimethicone, cetyl dimethicone, stearyl dimethicone, bis-stearyl dimethicone, and mixtures thereof.

4. Hydrophobic Components

The composition of the present invention may comprise from about 2% to about 75%, or from about 5% to about 35%, or from about 10% to about 30%, by weight of the composition, of a hydrophobic component. The hydrophobic component may be derived from animals, plants, or petroleum and may be natural or synthetic (i.e., man-made). Preferred hydrophobic components are substantially water-insoluble, more preferably essentially water-insoluble. Preferred hydrophobic components are those having a melting point of about 25° C. or less under about one atmosphere of pressure. Non-limiting examples of suitable hydrophobic components include those selected from the group consisting of mineral oil, petrolatum, esters, hydrocarbons, straight and branched chain hydrocarbons having from about 7 to about 40 carbon atoms, C1-C30 alcohol esters of C1-C30 carboxylic acids and of C2-C30 dicarboxylic acids, mono-, di- and tri-glycerides of C1-C30 carboxylic acids, alkylene glycol esters of C1-C30 carboxylic acids, propoxylated and ethoxylated derivatives, C1-C30 mono- and poly-esters of sugars and related materials, organopolysiloxane oils (polyalkylsiloxanes, cyclic polyalkylsiloxanes, trimethylsiloxysilicate, dimethiconols, polyalkylaryl siloxanes), vegetable oils and hydrogenated vegetable oils, animal fats and oils, silicone elastomers, and combinations thereof. These components are provided in further detail in U.S. Pat. No. 5,997,887 and U.S. Application Publication No. US 2005/0019356 A1.

### II. METHODS

The present invention further relates to a method for improving the deliverability of a cationic active in the presence of an anionic thickener. In one aspect, this may be accomplished by providing the personal-care composition described above and applying the composition to keratinous tissue in need of treatment. In another aspect, this may be accomplished by preparing an oil phase and a water phase; adding from about 0.001% to about 20% of a cationic active to either the oil phase or the water phase; adding from about 0.0001% to about 20% of an carboxylic acid to either the oil phase or the water phase; adding a base to either the oil phase or the water phase; mixing the water phase and the oil phase to form an emulsion; and adding to the emulsion from about 0.001% to about 20% of an anionic thickener; wherein the molar ratio of the base to the acid is at least about 0.70 and wherein the equivalent ratio of the anionic pairing agent to the cationic active is at least about 0.70. In one embodiment, the anionic pairing agent is pre-formed from the neutralization of an acid with a base. In another embodiment, the anionic pairing agent is formed in situ from the neutralization of an acid with a base.

The present invention further relates to a method for improving or regulating keratinous tissue condition. In one aspect, this may be accomplished by providing the personal-care composition described above and applying the composition to keratinous tissue in need of treatment. Conditions to be improved or regulated include increasing the luminosity or “glow” of the skin, reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, dryness, brittleness, and graying hair.

### III. EXAMPLES

The following describe non-limiting examples of the personal-care composition. The reported percentages indicate the weight of the component expressed as a percentage of the total weight of the personal-care composition. Each Example may comprise one or more of the optional ingredients in amounts also disclosed herein.

### Examples 1-5

Moisturizing oil-in-water lotions/creams are prepared by the following preparation method. In a suitable vessel, the Phase A components are combined and mixed with a suitable mixer (e.g., Tekmar RW20DZM) and heated with stirring to a temperature of about 70-80° C. and this temperature is maintained. In a separate suitable vessel, the Phase B components are combined and mixed with a suitable mixer and are heated with stirring to about 70-75° C. and this temperature is maintained. The Phase B mixture is then added to the Phase A mixture and mixed well so as to emulsify the combination. The emulsion of Phase A and B components is allowed to cool to about 60° C. and then the Phase D components are added to the emulsion with continuous mixing. The emulsion of Phase A, B, and D components is allowed to further cool to about 40° C., and then the Phase C and E components are added with mixing to the emulsion. The resulting emulsion is then milled using a suitable mill (e.g., with a Tekmar T-25) for about 5 minutes or until the product is uniform.

| | | | | | |
|---|---|---|---|---|---|
| Components | Ex. 1 | Ex. 2 | Ex. 3 | Ex. 4 | Ex. 5 |

Phase A: Water Phase

| | | | | | |
|---|---|---|---|---|---|
| Water | qs | qs | qs | qs | qs |
| Glycerin | 3 | 5 | 7 | 10 | 15 |
| Disodium EDTA | 0.1 | 0.1 | 0.05 | 0.1 | 0.1 |
| Methylparaben | 0.1 | 0.1 | 0.1 | 0.1 | 0.1 |
| Niacinamide | 2 | 0.5 | — | 3 | 5 |
| D-panthenol | 0.5 | 0.1 | — | 0.5 | 0.5 |
| Benzyl Alcohol | 0.25 | 0.25 | 0.25 | 0.25 | 0.25 |
| GLW75CAP-MP¹ | — | 0.5 | 0.5 | — | — |
| Palmitoyl Dipeptide-7 | 0.00055 | — | — | 0.0003 | — |
| N-acetyl glucosamine | 2 | — | 5 | — | 1 |
| 1,5-Pentanediol | — | — | 5 | 1 | 3 |
| Palmitoyl Pentapeptide-4 | — | 0.0003 | 0.001 | — | — |
| Hexamidine Diisethionate | — | — | 1 | 0.1 | 0.1 |
| Cetylpyridinium Chloride | 0.4 | 0.2 | — | — | — |
| Sodium Stearate | 0.34 | — | — | — | — |
| Triethanolamine | — | 0.54 | 0.55 | 0.05 | — |

Phase B: Oil Phase

| | | | | | |
|---|---|---|---|---|---|
| Tetrahydrocurcumin | — | 0.5 | — | — | — |
| Salicylic Acid | — | — | 0.5 | — | — |
| Isohexadecane | 3 | 3 | 3 | — | 3 |
| Octyl Salicylate | — | — | — | 5 | — |
| Octocrylene | — | — | — | 2.5 | — |
| Butyl | — | — | — | 3 | — |
| methoxydibenzoylmethane | | | | | |
| Homosalate | — | — | — | 5 | — |
| PPG15 Stearyl Ether | — | 5 | 8 | — | 2 |
| Isopropyl Isostearate | 5 | 2 | 1.3 | — | 1.3 |
| Sucrose Polyester | 0.7 | — | 0.7 | 1 | 0.7 |
| Undecylenoyl Phenylalanine | — | 1 | — | — | — |
| Phytosterol | — | — | 0.5 | — | 0.1 |
| Cetyl alcohol | 0.5 | 0.25 | 0.5 | 0.25 | 0.5 |
| Stearyl alcohol | 0.4 | 0.3 | 0.4 | 0.5 | 0.4 |
| Behenyl alcohol | 0.5 | 0.35 | 0.5 | 0.6 | 0.5 |
| PEG-100 stearate | 0.4 | 0.3 | 0.4 | 0.6 | 0.4 |
| Cetearyl glucoside | 0.1 | 0.1 | 0.1 | 1 | 0.1 |
| Oleic Acid | — | — | — | 0.1 | — |
| Stearic Acid | — | 0.16 | — | — | 0.1 |
| Triethanolamine | — | — | — | — | 0.05 |

Phase C: Retinoid Phase

| | | | | | |
|---|---|---|---|---|---|
| Retinyl Propionate | — | — | — | 0.3 | — |
| C12-C15 Alkyl Benozate | — | — | — | 5 | — |
| Vitamin E Acetate | — | — | — | 0.5 | — |

Phase D: Thickener

| | | | | | |
|---|---|---|---|---|---|
| Polyacrylamide/C13-14 | 2.5 | — | — | — | 3 |
| isoparaffin/laureth-7² | | | | | |
| Acrylates/C10-30 alkyl | — | 0.6 | 0.4 | 0.5 | — |
| acrylates crosspolymer³ | | | | | |

Phase E: Additional Ingredients

| | | | | | |
|---|---|---|---|---|---|
| Dimethicone/dimethiconol⁴ | — | 1 | 2 | 0.5 | 2 |
| Polymethylsilsequioxane⁵ | — | — | 0.25 | — | 1 |
| Nylon-12 | — | 0.5 | — | — | — |
| Prestige Silk Violet⁶ | — | — | — | — | 1 |
| Timiron Splendid Red⁷ | — | 1 | — | 2 | — |
| TOTAL | 100.00 | 100.00 | 100.00 | 100.00 | 100.00 |

¹75% aqueous TiO2 dispersion from Kobo, South Plainfield, NJ.
²Sepigel 305 from Seppic, Fairfield, NJ.
³Cosmedia ATH from Cognis, Ambler, PA.
⁴Dow Corning 1503 from Dow Corning, Midland, MI.
⁵Polymethylsilsequioxane as CF600 from Momentive Performance, Albany, NY.
⁶Prestige Silk Violet from Aston Chemicals Aylesbury, UK.
⁷Timiron Splendid Red from Merck KGaA, Germany.

### Examples 6-11

Moisturizing silicone-in-water serum/lotions are prepared according to the following preparation method. In a suitable vessel, the Phase A components are blended together with a suitable mixer (e.g., Tekmar model RW20DZM) and mixing is continued until all of the components are dissolved. Then, the Phase B components are blended together in suitable vessel and mixed until homogeneous. Phase C ingredients are mixed together and are milled using a suitable mill (e.g., Tekmar RW-20) for about 5 minutes. The Phase B components are then added to the Phase C mixture with mixing. Then, the Phase D components are added to the mixture of Phases B and C. The combination of Phase B, C and D components is added to Phase A and the resulting emulsion is milled (e.g., with a Tekmar T-25). Then, Phase E is slowly added to the emulsion with mixing. Phase F is added and mixed until the product is uniform.

| | | | | | | |
|---|---|---|---|---|---|---|
| Components | Ex. 6 | Ex. 7 | Ex. 8 | Ex. 9 | Ex. 10 | Ex. 11 |

Phase A: Water Phase

| | | | | | | |
|---|---|---|---|---|---|---|
| Water | qs | qs | qs | qs | qs | qs |
| Glycerin | 3 | 7 | 5 | 10 | 15 | 10 |
| Disodium EDTA | 0.1 | 0.1 | 0.05 | 0.1 | 0.1 | 0.1 |
| Niacinamide | 2 | 0.5 | — | 3 | 5 | 3 |
| D-panthenol | 0.5 | 0.1 | — | 0.5 | 1.5 | 0.5 |
| GLW75CAP-MP¹ | — | 0.4 | — | — | — | 0.4 |
| Ascorbyl Glucoside | — | — | 0.2 | — | — | — |
| Palmitoyl Dipeptide-7 | 0.00055 | — | 0.001 | 0.0003 | — | — |
| Hydrolyzed soy protein | — | 4 | — | — | — | — |
| N-acetyl glucosamine | 2 | — | — | — | 5 | — |
| 1,2-pentanediol | 1 | 2 | 2 | 3 | 1 | — |
| Palmitoyl Pentapeptide-4 | — | 0.0003 | — | 0.001 | — | — |
| Sodium Stearate | — | — | — | — | 0.20 | — |
| Hexamidine Diisethionate | | | 0.04 | 1 | 0.2 | 0.1 |
| Cetylpyridinium Chloride | 0.2 | 0.4 | — | — | — | — |
| Triethanolamine | 0.09 | 0.18 | 0.11 | — | — | — |

Phase B: Oil Phase

| | | | | | | |
|---|---|---|---|---|---|---|
| Oleic Acid | 0.16 | 0.32 | — | — | — | — |
| Stearic Acid | — | — | 0.04 | — | — | 0.1 |
| Triethanolamine | — | — | — | 0.54 | — | 0.05 |
| PPG-15 Stearyl Ether | 4 | 6 | 2 | 3 | 2 | 2 |
| Vitamin E Acetate | — | 0.5 | 0.1 | 0.1 | — | 0.1 |
| Brij 30 | 0.3 | 0.5 | 0.2 | 0.4 | 0.3 | 0.3 |
| Tetrahydrocurcumin | — | 0.1 | — | — | — | — |
| Octyl Salicylate | — | — | — | — | 5 | — |
| Octocrylene | — | — | — | — | 2.5 | — |
| Butly | — | — | — | — | 3 | — |
| methoxydibenzoylmethane | | | | | | |
| Homosalate | — | — | — | — | 5 | — |
| Salicylic Acid | — | — | — | 0.5 | — | — |
| Phytosterol | — | — | — | — | — | 0.1 |
| Undecylenoyl Phenylalanine | — | — | 0.2 | — | — | — |

Phase C: Silicone Phase

| | | | | | | |
|---|---|---|---|---|---|---|
| Cyclomethicone D5 | 10 | 5 | 5 | 10 | 7.5 | 10 |
| Dow corning 9040 | — | 10 | 5 | 5 | 7.5 | 5 |
| Dimethicone/dimethiconol² | 1 | — | — | — | — | — |
| Dimethicone 50 csk | 1 | — | — | — | — | — |

Phase D: Retinoid

| | | | | | | |
|---|---|---|---|---|---|---|
| Retinyl Propionate | — | — | — | — | 0.3 | — |
| C12-C15 Alkyl Benozate | — | — | — | — | 5 | — |

Phase E: Thickener

| | | | | | | |
|---|---|---|---|---|---|---|
| Polyacrylamide/C13-14 | 2.5 | 2.5 | — | — | — | 3 |
| isoparaffin/laureth-7³ | | | | | | |
| Acrylates/C10-30 alkyl | — | — | 0.6 | 0.4 | 0.5 | — |
| acrylates crosspolymer⁴ | | | | | | |

Phase F: Additional Ingredients

| | | | | | | |
|---|---|---|---|---|---|---|
| Polymethylsilsequioxane⁵ | 0.5 | 0.5 | 1 | 1 | 1 | 0.5 |
| Polyethylene⁶ | — | 0.5 | 0.5 | 1 | — | — |
| TOTAL | 100.00 | 100.00 | 100.00 | 100.00 | 100.00 | 100.00 |

¹75% aqueous TiO2 dispersion from Kobo, South Plainfield, NJ.
²Dow Corning 1503 from Dow Corning, Midland, MI.
³Sepigel 305 from Seppic, Fairfield, NJ.
⁴Cosmedia ATH from Cognis, Ambler, PA.
⁵Polymethylsilsequioxane as CF600 from Momentive Performance, Albany, NY.
⁶Microthene FN 510-00 from Lyondel Basell, Rotterdam, Netherlands.

### Examples 12-16

Oil-in-water mousses are prepared according to the following preparation method. In a suitable vessel, the Phase A components are combined and mixed until uniform. In a separate suitable vessel, the Phase B components are combined and mixed until uniform. The Phase B mixture is then added to the Phase A mixture and the resulting emulsion is milled (e.g., with a Tekmar T-25). The Phase C components and then the Phase D components are added to the emulsion while stirring. Once the composition is uniform, the product is poured into suitable containers. The product and Phase E are added into an aerosol container. The aerosol container is then sealed.

| | | | | | |
|---|---|---|---|---|---|
| Components | Ex. 12 | Ex. 13 | Ex. 14 | Ex. 15 | Ex. 16 |

Phase A: Water Phase

| | | | | | |
|---|---|---|---|---|---|
| Water | qs | qs | qs | qs | qs |
| Glycerin | 3 | 5 | 7 | 10 | 15 |
| Disodium EDTA | 0.1 | 0.1 | 0.05 | 0.1 | 0.1 |
| Methylparaben | 0.1 | 0.1 | 0.1 | 0.1 | 0.1 |
| Niacinamide | 2 | 0.5 | — | 3 | 5 |
| D-panthenol | 0.5 | 0.1 | — | 0.5 | 1.5 |
| Benzyl Alcohol | 0.25 | 0.25 | 0.25 | 0.25 | 0.25 |
| GLW75CAP-MP¹ | — | 0.5 | 0.5 | — | — |
| Palmitoyl Dipeptide-7 | 0.00055 | — | 0.001 | — | — |
| N-acetyl glucosamine | 2 | 1 | 2 | 2 | 1 |
| Palmitoyl Pentapeptide-4 | — | 0.001 | — | 0.0003 | — |
| 1,5-Pentanediol | 0.5 | 2 | 1 | 3 | 0.5 |
| Hexamidine Diisethionate | | | 0.06 | 1 | 0.1 |
| Cetylpyridinium Chloride | 0.2 | 0.4 | | | |
| Sodium Stearate | — | — | — | — | 0.10 |
| Triethanolamine | 0.09 | 0.23 | — | — | — |

Phase B: Oil Phase

| | | | | | |
|---|---|---|---|---|---|
| Salicylic Acid | — | — | — | 0.5 | — |
| Isohexadecane | 3 | 3 | 3 | 1 | 3 |
| PPG15 Stearyl Ether | 8 | 10 | — | 8 | 15 |
| Isopropyl Isostearate | 1 | 0.5 | 1.3 | 1.5 | 1.3 |
| Sucrose polyester | 0.7 | — | 0.7 | 1 | 0.7 |
| Undecylenoyl Phenylalanine | — | 0.5 | — | — | — |
| Tetrahydrocucurmin | — | 1 | — | 0.5 | — |
| Octyl Salicylate | — | — | — | — | 5 |
| Octocrylene | — | — | — | — | 2.5 |
| Butly | — | — | — | — | 3 |
| methoxydibenzoylmethane | | | | | |
| Homosalate | — | — | — | — | 5 |
| Phytosterol | — | — | 0.5 | — | 0.1 |
| Cetyl alcohol | 0.4 | 0.3 | 0.4 | 0.5 | 0.4 |
| Stearyl alcohol | 0.5 | 0.35 | 0.5 | 0.6 | 0.5 |
| Behenyl alcohol | 0.4 | 0.3 | 0.4 | 0.5 | 0.4 |
| PEG-100 stearate | 0.1 | 0.1 | 0.1 | 0.2 | 0.1 |
| Cetearyl glucoside | 0.1 | 0.1 | 0.1 | 0.25 | 0.1 |
| Oleic Acid | 0.16 | — | — | — | — |
| Stearic Acid | — | — | 0.06 | — | — |
| Triethanolamine | — | — | 0.032 | 0.54 | — |

Phase C: Thickener

| | | | | | |
|---|---|---|---|---|---|
| Polyacrylamide/C13-14 | 2.5 | — | — | — | 3 |
| isoparaffin/laureth-7² | | | | | |
| Acrylates/C10-30 alkyl | — | 0.6 | 0.4 | 0.5 | — |
| acrylates crosspolymer³ | | | | | |

Phase D: Additional Ingredients

| | | | | | |
|---|---|---|---|---|---|
| Dimethicone/dimethiconal⁴ | — | 1 | 2 | 0.5 | 2 |
| Polymethylsilsequioxane⁵ | — | — | 0.25 | — | 1 |
| Nylon-12 | — | 0.5 | — | — | — |
| Prestige Silk Violet⁶ | — | — | — | — | 1 |
| Timiron Splendid Red⁷ | — | 1 | — | 2 | — |

Phase E: Propellant Phase

| | | | | | |
|---|---|---|---|---|---|
| 152 A HFC Propellant | 3 | 4 | 2 | 3 | 2 |
| A-70 Propellant | 3 | 2 | 4 | 3 | 4 |
| TOTAL | 100.00 | 100.00 | 100.00 | 100.00 | 100.00 |

¹75% aqueous TiO2 dispersion from Kobo, South Plainfield, NJ.
²Sepigel 305 from Seppic, Fairfield, NJ.
³Cosmedia ATH from Cognis, Ambler, PA.
⁴Dow Corning 1503 from Dow Corning, Midland, MI.
⁵Polymethylsilsequioxane as CF600 from Momentive Performance, Albany, NY.
⁶Prestige Silk Violet from Aston Chemicals Aylesbury, UK.
⁷Timiron Splendid Red from Merck KGaA, Germany.

### Examples 17-22

Silicone-in-water mousses are prepared according to the following preparation method. In a suitable vessel, the Phase A components are combined and mixed until uniform. In a separate suitable vessel, the Phase B components are combined and mixed until uniform. The Phase B mixture is added to the Phase A mixture and the resulting emulsion is milled (e.g., with a Tekmar T-25). The Phase C components and then the Phase D components are added to the emulsion while stirring. Once the composition is uniform, the product is poured into suitable containers. The product and Phase E are added into an aerosol container. The aerosol container is then sealed.

| | | | | | | |
|---|---|---|---|---|---|---|
| Components | Ex. 17 | Ex. 18 | Ex. 19 | Ex. 20 | Ex. 21 | Ex. 22 |

Phase A: Water Phase

| | | | | | | |
|---|---|---|---|---|---|---|
| Water | qs | qs | qs | qs | qs | qs |
| Glycerin | 3 | 5 | 7 | 10 | 15 | 10 |
| Disodium EDTA | 0.1 | 0.1 | 0.05 | 0.1 | 0.1 | 0.1 |
| Niacinamide | 2 | 0.5 | — | 3 | 5 | 3 |
| D-panthenol | 0.5 | 0.1 | — | 0.5 | 1.5 | 0.5 |
| GLW75CAP-MP¹ | — | 0.4 | — | — | — | — |
| Ascorbyl Glucoside | — | — | — | — | — | 1 |
| Palmitoyl-Dipeptide-7 | 0.0003 | — | 0.00055 | 0.00055 | — | 0.00055 |
| Palmitoyl Pentapeptide-4 | — | 0.0003 | — | — | 0.001 | — |
| Hydrolyzed Soy Protein | — | 1 | — | — | — | — |
| N-acetyl Glucosamine | 2 | — | 2 | — | 5 | — |
| 1,2-Hexanediol | 0.2 | 2 | 1 | — | 0.5 | — |
| 1,2-Pentanediol | 0.5 | — | 1 | 3 | — | 3 |
| Hexamidine Diisethionate | 0.1 | — | — | 0.06 | 0.9 | 0.1 |
| Cetylpyridinium Chloride | — | 0.2 | 0.4 | — | — | — |
| Triethanolamine | 0.05 | 0.085 | 0.27 | — | — | — |

Phase B: Silicone/Oil Phase

| | | | | | | |
|---|---|---|---|---|---|---|
| Cyclomethicone D5 | 10 | 5 | 5 | 10 | 7.5 | 10 |
| Dow Corning 9040 | — | 10 | 5 | 5 | 7.5 | 5 |
| Dimethicone/dimethiconol² | 1 | — | — | — | — | — |
| Dimethicone 50 csk | 1 | — | — | — | — | — |
| Salicylic Acid | — | — | 0.25 | — | — | — |
| Phytosterol | — | — | — | 0.2 | — | 0.1 |
| Brij 30 | 0.5 | 0.5 | 1 | 1 | 0.3 | 0.3 |
| PPG-15 Stearyl Ether | 2 | 4 | 4 | 4 | 3 | 2 |
| Tetrahydrocurcumin | — | 0.5 | — | 0.1 | — | — |
| Undecylenoyl Phenylalanine | — | — | — | — | 1 | — |
| Vitamin E Acetate | — | 0.5 | 0.1 | 0.1 | — | 0.1 |
| Oleic Acid | 0.1 | 0.16 | — | | — | — |
| Stearic Acid | — | — | — | 0.056 | — | 0.1 |
| Triethanolamine | — | — | — | 0.03 | 0.45 | 0.05 |

Phase C: Thickener

| | | | | | | |
|---|---|---|---|---|---|---|
| Polyacrylamide/C13-14 | 2.5 | 2.5 | — | — | — | 3 |
| isoparaffin/laureth-7³ | | | | | | |
| Acrylates/C10-30 alkyl | — | — | 0.6 | 0.4 | 0.5 | — |
| acrylates crosspolymer⁴ | | | | | | |

Phase D: Additional Ingredients

| | | | | | | |
|---|---|---|---|---|---|---|
| Polymethylsilsequioxane⁵ | 0.5 | 0.5 | 1 | 1 | 1 | 0.5 |
| Polyethylene⁶ | — | 0.5 | 0.5 | 1 | — | — |
| Silica⁷ | — | — | 1 | 0.5 | — | — |

Phase E: Propellant Phase

| | | | | | | |
|---|---|---|---|---|---|---|
| 152 A HFC Propellant | 3 | 3 | 4 | 2 | 3 | 2 |
| A-70 Propellant | 3 | 3 | 2 | 4 | 3 | 4 |
| TOTAL | 100.00 | 100.00 | 100.00 | 100.00 | 100.00 | 100.00 |

¹75% aqueous TiO2 dispersion from Kobo, South Plainfield, NJ.
²Dow Corning 1503 from Dow Corning, Midland, MI.
³Sepigel 305 from Seppic, Fairfield, NJ.
⁴Cosmedia ATH from Cognis, Ambler, PA.
⁵Polymethylsilsequioxane as CF600 from Momentive Performance, Albany NY.
⁶Microthene FN 510-00 from Lyondel Basell, Rotterdam, Netherlands.
⁷Cab-o-Sil TS-720 from Cabot,Tuscola, IL

### IV. COMPARATIVE EXAMPLES

The following is a comparison between examples falling within the present invention and comparative examples using conventional materials. The reported percentages indicate the weight of the component expressed as a percentage of the total weight of the personal-care composition. The Comparative Examples may comprise one or more optional ingredients in amounts also disclosed herein. The Comparative Examples may be prepared by the methods used in preparation of the Examples above from the following components. The weight percent of cationic active in each phase (i.e., oil and water) of a finished product, by weight of the total product, may be determined through high performance liquid chromatography (HPLC) with UV detection at 245 nm.

To determine the amount of cationic active in the oil phase of a finished product, weigh about 4.5 g of product in an ultracentrifuge tube. Keep the total weight of the product sample plus tube about the same (+/−10 mg). Centrifuge the product sample for 48 hours at 60,000 rpm in a Beckman Coulter Optima L-100 XP Ultracentrifuge or equivalent. The product sample will separate into distinct phases. Separate the different phases with syringes and knives. Weigh the samples into glass vials according to the analytical method. Add 6 ml of methanol with internal standard. Vortex to completely disperse the product sample. Add 5 ml of diluents (comprising 9% methanol, 20 mM formic acid, and 30 mM ammonium acetate) and mix thoroughly. Filter product samples into HPLC vials. Analyze the cationic active by HPLC with UV detection.

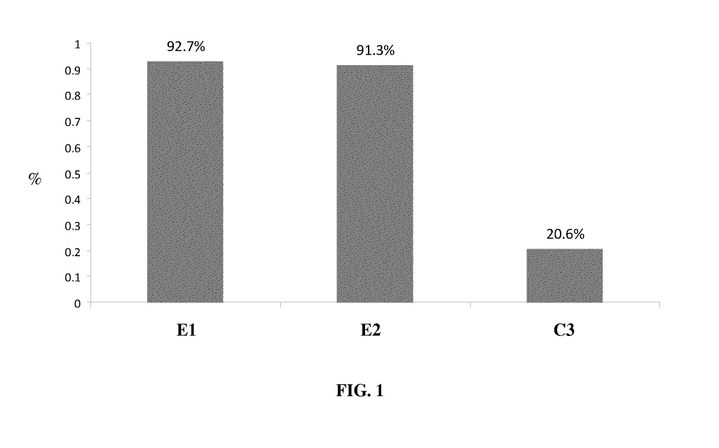

Examples E1 and E2 highlight the increase in availability of a cationic active in the oil phase when an anionic pairing agent is included within a formulation comprising an anionic thickener (E1-E2, falling within the present invention). In E1 and E2, 0.028% base is added to the composition which is believed to completely neutralize 0.10% stearic acid in situ (molar ratio of base:acid is 1:1). In addition, the amount of stearic acid initially present and subsequent sodium sterarate formed is at a concentration sufficient to completely complex with all the Hexamidine Diisethionnate present (equivalent ratio of anionic pairing agent:cationic active is 1:1). The use of only 0.0176% base in C3 results in not all of the 0.10% stearic acid being neutralized and subsequently less than 100% of the Hexamidine Diisethionate will ion-pair with the neutralized stearic acid. The equivalent ratio of anionic pairing agent:cationic active that results from only partial neutralization of stearic acid is 0.67:1 rather than the preferred 1:1 equivalent ratio. As can be calculated and illustrated in FIG. 1, further described below, in C3 only 63% of the stearic acid is complexed. Net, the level of in situ formed anionic pairing agent, sodium stearate, is not sufficient to ion-pair with all of the Hexamidine Diisethionate present. In C3, less anionic pairing agent formed than is preferred. C3 falls outside the present invention.

| Components | E1 | E2 | C3 |
|---|---|---|---|
| Water Phase | | | |
| Purified Water | qs | qs | qs |
| Glycerin | 10.00 | 10.00 | 10.00 |
| Disodium EDTA | 0.10 | 0.10 | 0.10 |
| D-panthenol | 1.00 | 1.00 | 1.00 |
| Benzyl Alcohol | 0.25 | 0.25 | 0.25 |
| Sodium Hydroxide 50% | 0.028 | — | 0.0176 |
| Hexamidine Diisethionate | 0.10 | 0.10 | 0.10 |
| Oil Phase | | | |
| Isopropyl Isostearate | 1.50 | 1.50 | 1.50 |
| Isohexadecane | 3.00 | 3.00 | 3.00 |
| Vitamin E Acetate | 0.25 | 0.25 | 0.25 |
| Cetyl Alcohol | 0.515 | 0.515 | 0.515 |
| Stearyl Alcohol | 0.641 | 0.641 | 0.641 |
| Behenyl Alcohol | 0.42 | 0.42 | 0.42 |
| PEG-100 Stearate | 0.10 | 0.10 | 0.10 |
| Cetearyl Glucoside | 0.20 | 0.20 | 0.20 |
| Ethylparaben | 0.07 | 0.07 | 0.07 |
| Sodium Hydroxide 50% | — | 0.028 | — |
| Stearic Acid | 0.10 | 0.10 | 0.10 |
| Thickener | | | |
| Polyacrylamide/C13-14 | 1.50 | 1.50 | 1.50 |
| Isoparaffin/laureth-7¹ | | | |
| Additional Ingredients | | | |
| Dimethicone/Dimethiconol² | 1.00 | 1.00 | 1.00 |
| Total | 100.00 | 100.00 | 100.00 |
| Percent cationic active present in | 92.7 | 91.3 | 20.6 |
| oil phase of final product (based on | | | |
| initial active wt %) | | | |
| Molar ratio of base to acid | 1:1 | 1:1 | 0.63:1 |

¹Sepigel 305 from Seppic, Fairfield, NJ.
²Dow Corning 1503 from Dow Corning, Midland, MI.

The results of the Comparative Examples are provided in FIG. 1, describing the analytically measured cationic active in the oil phase. Comparative Examples C3, E1, and E2 are displayed along the x-axis. The y-axis, %, represents the percent of cationic active in the oil phase. As may be seen, there is a significant jump from C3, with 20.6% of the cationic active in the oil phase, to E1 and E2, with 92.7% and 91.3%, respectively, of the cationic active in the oil phase. The figure demonstrates the unexpected benefit of adding more than 63% of the anionic pairing agent: there is an unexpected increase in the amount of cationic active in the oil phase.

As previously outlined in the Background, it is not desirable to have the cationic active reside in the water phase. Applicants have surprisingly found that the complexing of the cationic active with the anionic pairing agent significantly increases the presence of the cationic active in the oil phase. This prevents the cationic active from interacting with the anionic thickener. Thus, there is better bioavailability and efficacy of the cationic active.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal-care composition in the form of an oil-in-water emulsion comprising:
a) from about 0.0001% to about 20% of an anionic pairing agent;
b) from about 0.001% to about 20% of a cationic active; and
c) from about 0.001% to about 20% of an anionic thickener;
wherein the anionic pairing agent is pre-formed from the neutralization of an acid with a base, wherein the molar ratio of the base to the acid is at least about 0.70, and wherein the acid is selected from the group consisting of carboxylic acids, alkyl or aryl sulfonic acids, and salts, derivatives, and mixtures thereof.

2. The personal-care composition of claim 1, wherein the acid is selected from the group consisting of oleic acid, stearic acid, propionic acid, hexanoic acid, benzoic acid, octadecenedioic acid, retinoic acid, salicylic acid, glycyrrhetinic acid, undecylenoyl phenylalanine, and combinations thereof.

3. The personal-care composition of claim 1, wherein the base is selected from the group consisting of triethylamine, triethanolomine, sodium hydroxide, potassium hydroxide, ammonium hydroxide, calcium hydroxide, magnesium hydroxide, and combinations thereof.

4. The personal-care composition of claim 1, wherein the cationic active is selected from the group consisting of hexamidine compounds, cetylpyridinium chloride, amino acids, and combinations thereof.

5. The personal-care composition of claim 1, wherein the anionic thickener is selected from the group consisting of sodium polyacrylate, sodium polyacrylate/hydrogentated polydecence/PPG-5 Laureth-5, polyacrylamide/C13-14/laureth-7, sodium polyacrylate/C13-14 isoparaffin/trideceth-6, and combinations thereof.

6. The personal-care composition of claim 1, wherein the molar ratio of the base to the acid is at least about 0.80.

7. The personal-care composition of claim 1, wherein the molar ratio of the base to the acid is at least about 0.90.

8. The personal-care composition of claim 1, wherein the molar ratio of the base to the acid is about 1:1.

9. A method for improving deliverability of a cationic active in the presence of an anionic thickener, comprising the steps of:
a) providing the personal-care composition of claim 1; and
b) applying the composition to keratinous tissue in need of treatment.

10. A method for improving or regulating keratinous tissue condition, comprising the steps of:
a) providing the personal-care composition of claim 1; and
b) applying the composition to keratinous tissue in need of treatment.

11. A personal-care composition in the form of an oil-in-water emulsion comprising:
a) from about 0.0001% to about 20% of an anionic pairing agent;
b) from about 0.001% to about 20% of a cationic active; and
c) from about 0.001% to about 20% of an anionic thickener;
wherein the anionic pairing agent is pre-formed from the neutralization of an acid with a base, wherein the equivalent ratio of the anionic pairing agent to the cationic active is at least about 0.70, and wherein the acid is selected from the group consisting of carboxylic acids, alkyl or aryl sulfonic acids, and salts, derivatives, and mixtures thereof.

12. The personal-care composition of claim 11, wherein the acid is selected from the group consisting of oleic acid, stearic acid, propionic acid, hexanoic acid, benzoic acid, octadecenedioic acid, retinoic acid, salicylic acid, glycyrrhetinic acid, undecylenoyl phenylalanine, and combinations thereof.

13. The personal-care composition of claim 11, wherein the base is selected from the group consisting of triethylamine, triethanolomine, sodium hydroxide, potassium hydroxide, ammonium hydroxide, calcium hydroxide, magnesium hydroxide, and combinations thereof.

14. The personal-care composition of claim 11, wherein the cationic active is selected from the group consisting of hexamidine compounds, cetylpyridinium chloride, amino acids, and combinations thereof.

15. The personal-care composition of claim 11, wherein the anionic thickener is selected from the group consisting of sodium polyacrylate, sodium polyacrylate/hydrogentated polydecence/PPG-5 Laureth-5, polyacrylamide/C13-14/laureth-7, sodium polyacrylate/C13-14 isoparaffin/trideceth-6, and combinations thereof.

16. A method for improving deliverability of a cationic active in the presence of an anionic thickener, comprising the steps of:
a) providing the personal-care composition of claim 11; and
b) applying the composition to keratinous tissue in need of treatment.

17. A method for improving or regulating keratinous tissue condition, comprising the steps of:
a) providing the personal-care composition of claim 11; and
b) applying the composition to keratinous tissue in need of treatment.

18. A personal-care composition in the form of an oil-in-water emulsion comprising:
a) from about 0.0001% to about 20% of an anionic pairing agent;
b) from about 0.001% to about 20% of a cationic active; and
c) from about 0.001% to about 20% of an anionic thickener;
wherein the anionic pairing agent is formed in situ from the neutralization of an acid with a base, wherein the molar ratio of the base to the acid is at least about 0.70, and wherein the acid is selected from the group consisting of carboxylic acids, alkyl or aryl sulfonic acids, and salts, derivatives, and mixtures thereof.

19. The personal-care composition of claim 18, wherein the acid is selected from the group consisting of oleic acid, stearic acid, propionic acid, hexanoic acid, benzoic acid, octadecenedioic acid, retinoic acid, salicylic acid, glycyrrhetinic acid, undecylenoyl phenylalanine, and combinations thereof.

20. The personal-care composition of claim 18, wherein the base is selected from the group consisting of triethylamine, triethanolomine, sodium hydroxide, potassium hydroxide, ammonium hydroxide, calcium hydroxide, magnesium hydroxide, and combinations thereof.

21. The personal-care composition of claim 18, wherein the cationic active is selected from the group consisting of hexamidine compounds, cetylpyridinium chloride, amino acids, and combinations thereof.

22. The personal-care composition of claim 18, wherein the anionic thickener is selected from the group consisting of sodium polyacrylate, sodium polyacrylate/hydrogentated polydecence/PPG-5 Laureth-5, polyacrylamide/C13-14/laureth-7, sodium polyacrylate/C13-14 isoparaffin/trideceth-6, and combinations thereof.

23. The personal-care composition of claim 18, wherein the molar ratio of the base to the acid is at least about 0.80.

24. The personal-care composition of claim 18, wherein the molar ratio of the base to the acid is at least about 0.90.

25. The personal-care composition of claim 18, wherein the molar ratio of the base to the acid is about 1:1.

26. A method for improving deliverability of a cationic active in the presence of an anionic thickener, comprising the steps of:
a) providing the personal-care composition of claim 18; and
b) applying the composition to keratinous tissue in need of treatment.

27. A method for improving or regulating keratinous tissue condition, comprising the steps of:
a) providing the personal-care composition of claims 18; and
b) applying the composition to keratinous tissue in need of treatment.

28. A personal-care composition in the form of an oil-in-water emulsion comprising:
a) from about 0.0001% to about 20% of an anionic pairing agent;
b) from about 0.001% to about 20% of a cationic active; and
c) from about 0.001% to about 20% of an anionic thickener;
wherein the anionic pairing agent is formed in situ from the neutralization of an acid with a base, wherein the equivalent ratio of the anionic pairing agent to the cationic active is at least about 0.70, and wherein the acid is selected from the group consisting of carboxylic acids, alkyl or aryl sulfonic acids, and salts, derivatives, and mixtures thereof.

29. The personal-care composition of claim 28, wherein the acid is selected from the group consisting of oleic acid, stearic acid, propionic acid, hexanoic acid, benzoic acid, octadecenedioic acid, retinoic acid, salicylic acid, glycyrrhetinic acid, undecylenoyl phenylalanine, and combinations thereof.

30. The personal-care composition of claim 28, wherein the base is selected from the group consisting of triethylamine, triethanolomine, sodium hydroxide, potassium hydroxide, ammonium hydroxide, calcium hydroxide, magnesium hydroxide, and combinations thereof.

31. The personal-care composition of claim 28, wherein the cationic active is selected from the group consisting of hexamidine compounds, cetylpyridinium chloride, amino acids, and combinations thereof.

32. The personal-care composition of claim 28, wherein the anionic thickener is selected from the group consisting of sodium polyacrylate, sodium polyacrylate/hydrogentated polydecence/PPG-5 Laureth-5, polyacrylamide/C13-14/laureth-7, sodium polyacrylate/C13-14 isoparaffin/trideceth-6, and combinations thereof.

33. A method for improving deliverability of a cationic active in the presence of an anionic thickener, comprising the steps of:
a) providing the personal-care composition of claim 28; and
b) applying the composition to keratinous tissue in need of treatment.

34. A method for improving or regulating keratinous tissue condition, comprising the steps of:
a) providing the personal-care composition of claim 28; and
b) applying the composition to keratinous tissue in need of treatment.

35. A method for improving deliverability of a cationic active in the presence of an anionic thickener, comprising the steps of:
a) preparing an oil phase and a water phase;
b) adding from about 0.001% to about 20% of a cationic active to either the oil phase or the water phase;
c) adding from about 0.0001% to about 20% of an carboxylic acid to either the oil phase or the water phase;
d) adding a base to either the oil phase or the water phase;
e) mixing the water phase and the oil phase to form an emulsion; and
f) adding to the emulsion from about 0.001% to about 20% of an anionic thickener;
wherein the molar ratio of the base to the acid is at least about 0.70, and wherein the equivalent ratio of the anionic pairing agent to the cationic active is at least about 0.70.

36. The method of claim 35, wherein the anionic pairing agent is pre-formed from the neutralization of an acid with a base.

37. The method of claim 35, wherein the anionic pairing agent is formed in situ from the neutralization of an acid with a base.

38. The method of claim 35, wherein the carboxylic acid is selected from the group consisting of oleic acid, stearic acid, propionic acid, hexanoic acid, benzoic acid, octadecenedioic acid, retinoic acid, salicylic acid, glycyrrhetinic acid, undecylenoyl phenylalanine, and combinations thereof.

39. The method of claim 35, wherein the base is selected from the group consisting of triethylamine, triethanolomine, sodium hydroxide, potassium hydroxide, ammonium hydroxide, calcium hydroxide, magnesium hydroxide, and combinations thereof.

40. The method of claim 35, wherein the cationic active is selected from the group consisting of hexamidine compounds, cetylpyridinium chloride, amino acids, and combinations thereof.

41. The method of claim 35, wherein the anionic thickener is selected from the group consisting of sodium polyacrylate, sodium polyacrylate/hydrogentated polydecence/PPG-5 Laureth-5, polyacrylamide/C13-14/laureth-7, sodium polyacrylate/C13-14 isoparaffin/trideceth-6, and combinations thereof.

42. A personal-care composition in the form of an oil-in-water emulsion comprising:
a) from about 0.1% to about 5% of an anionic pairing agent;
b) from about 0.01% to about 1% of a hexamidine diisethionate; and
c) from about 0.1% to about 5% of a thickener selected from the group consisting of polyacrylamide/C13-14/laureth-7, sodium polyacrylate/C13-14 isoparaffin/trideceth-6, and combinations thereof;
wherein the anionic pairing agent is pre-formed from the neutralization of an acid with a base, wherein the molar ratio of the base to the acid is at least about 0.70, wherein the equivalent ratio of the anionic pairing agent to the cationic active is at least about 0.70, and wherein the acid is selected from the group consisting of stearic acid, oleic acid, and mixtures thereof.

43. A personal-care composition in the form of an oil-in-water emulsion comprising:
a) from about 0.1% to about 5% of an anionic pairing agent;
b) from about 0.01% to about 1% of a hexamidine diisethionate; and
c) from about 0.1% to about 5% of a thickener selected from the group consisting of polyacrylamide/C13-14/laureth-7, sodium polyacrylate/C13-14 isoparaffin/trideceth-6, and combinations thereof;
wherein the anionic pairing agent is formed in situ from the neutralization of an acid with a base, wherein the molar ratio of the base to the acid is at least about 0.70, wherein the equivalent ratio of the anionic pairing agent to the cationic active is at least about 0.70, and wherein the acid is selected from the group consisting of stearic acid, oleic acid, and mixtures thereof.
